(19) United States
(12) Patent Application Publication
(10) Pub. No.: US 2009/0280038 A1
(43) Pub. Date: Nov. 12, 2009

(54) Fresh scent flowers

(76) Inventors: Guy Michael Waltz, II, Greenbrae, CA (US); Daniel Conan Fitzgerald, San Rafael, CA (US)

Correspondence Address: Guy Waltz II, 807 Via Casitas, Greenbrae, CA, 94904, US

(21) Appl. No.: 12/072,275

(22) Filed: May 9, 2008

(51) Int. Cl. A61L 9/12

(52) U.S. Cl. 422/123

(57) ABSTRACT

Fresh Scent Flowers (Patent Pending) is a faux floral arrangement consisting of plastic, silk, and an air freshener. The pot holding the flowers is made of plastic. The stems for the flowers are plastic and the petals of the flowers are silk. The air freshener is small and compact, fitting neatly underneath the flowers in the flower pot, with air vents at the base of the flowers to allow the aroma to flow freely from the plant/flowers. The air freshener will run on replaceable batteries and replaceable scent cartridges. These can be easily replaced by removing the bottom of the pot which will give access to the air freshener. This as well, will have an on/off switch.

Fresh Scent Flowers (Patent Pending), an air freshener in the form of a faux floral arrangement, is made of a plastic flower pot with plastic stems and silk flower petals. Also, it contains, in the flower pot, an air freshener which runs on replaceable batteries and replaceable scent cartridges. The batteries provide the energy for the air freshener and the scent cartridges provide the aroma for the air freshener. It also has air vents for the aroma from the scent cartridges to permeate the surrounding area and an on and off switch to turn the air freshener on and off at will.

### BACKGROUND

The ornamental design for an air freshener device.

This invention pertains to aesthetically pleasing products, such as faux floral arrangements (which are visually pleasing) and air fresheners (which are aromatically pleasing) it combines both, solving the problem of faux flowers which look like real flowers but do not smell like them and air fresheners which smell good but are unsightly and obnoxious looking. References: D567,924, D567,923, D567,922, D567,921, D567,920, D567,918, D562,441, D561,883, D561,882, D561,318, D559,965, D559,373, D558,320, D557,785, D556,313, D556,311, Air fresheners and air freshener devices.

### BRIEF DESCRIPTION OF DRAWING

FIG. 1—Faux/Silk Flower Petals

FIG. 2—Faux Flower Stems

FIG. 3—Air vents for air freshener

FIG. 4—Air Freshener

FIG. 4A Batteries

FIG. 4B Scent cartridges

FIG. 4C On/Off Switch

FIG. 5—Flower pot for faux flowers

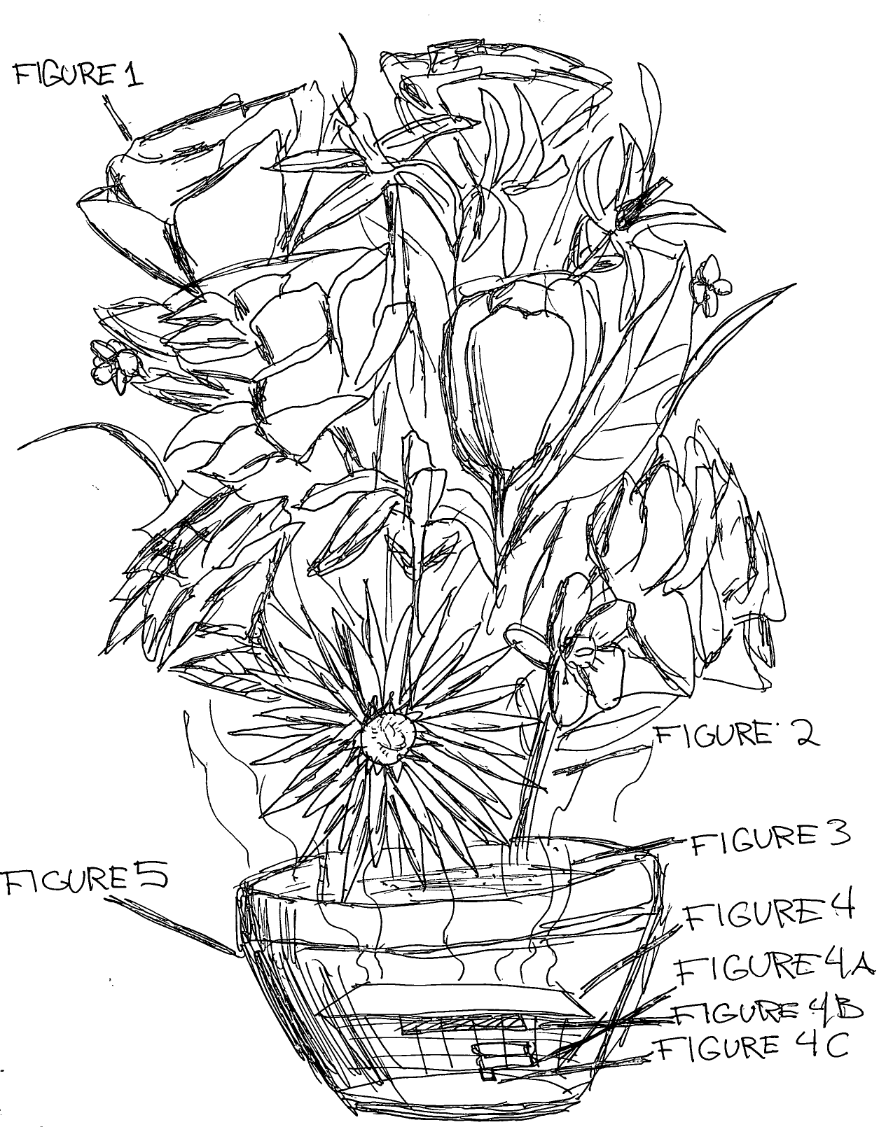

### DETAILED DESCRIPTION

Fresh Scent Flowers (Patent Pending) is in itself an air freshener with a unique decorative twist. It is fashioned in the appearance of a beautiful faux floral arrangement yet resembling normal faux floral arrangements, visually pleasing and soft on the eyes. However; it also has the refreshing fragrance of your standard air freshener up till now, pleasing both the eyes and the nose at the same time consequently; enhancing the experience.

It starts at the base, which is just a plastic flower pot with the advantage of a plastic panel with an on/off switch to turn the air freshener on and off. The air freshener is in the shape of a cube made of plastic and has openings to fit the batteries and the air freshener cartridge inside. This exclusive invention has air vents at top of the pot and/or at base of the flowers to allow the aroma to freely flow throughout the room in which the arrangement is placed. At the top of the pot, (i.e., where the air vents are located) are the plastic stems which are tipped with silk flower petals leaving you with Fresh Scent+flowers. (Patent Pending)

1. An air freshener of the type comprising of a faux floral arrangement of material having said air freshener in base of said faux floral arrangement, the improvement wherein said air freshener has said faux floral arrangement built around said air freshener.

2. The closure of claim 1 wherein said air freshener is powered by means of said batteries and said scent cartridges.

3. The closure of claim 1 wherein said faux floral arrangement has said air vents at base of said faux flower stems as means to permeate surrounding area with said scent cartridge aroma.

4. The closure of claim 1 wherein said faux floral arrangement is comprised of said flower pot, said stems, said flowers, and said air freshener.
